(19) United States
(12) Patent Application Publication
(10) Pub. No.: US 2023/0051268 A1
(43) Pub. Date: Feb. 16, 2023

(54) USE OF LEMBOREXANT FOR TREATING INSOMNIA

(71) Applicant: Eisai R&D Management Co., Ltd., Tokyo (JP)

(72) Inventors: Andrew SATLIN, Short Hills, NJ (US); Margaret MOLINE, White Plains, NY (US); Ishani LANDRY, Woodcliff Lake, NJ (US)

(21) Appl. No.: 17/757,672

(22) PCT Filed: Dec. 18, 2020
(86) PCT No.: PCT/US2020/065891

§ 371 Date: Jun. 17, 2022

Related U.S. Application Data
(60) Provisional application No. 62/951,638, filed on Dec. 20, 2019.

(51) Int. Cl. A61K 31/506; A61K 9/00

(52) U.S. Cl. CPC A61K 31/506; A61K 9/0053

(57) ABSTRACT

Disclosed are methods for treating insomnia, comprising administering orally a dosage form comprising Lemborexant or a pharmaceutically acceptable salt thereof to a patient in need thereof at a single daily dose ranging from 5 mg to 10 mg of lemborexant or an equivalent dose of a pharmaceutically acceptable salt thereof, provided that the maximum dose is 5 mg once per day of lemborexant or an equivalent dose of a pharmaceutically acceptable salt thereof when the patient has moderate hepatic impairment classified in Child-Pugh class B under Child-Pugh Classification.

The present application claims the benefit of priority to U.S. Provisional Application No. 62/951,638 filed Dec. 20, 2020; which is incorporated herein by reference.

The present disclosure relates to methods for treating insomnia.

Two neuropeptides, orexin-A (OX-A, a peptide consisting of 33 amino acids) and orexin-B (OX-B, a peptide consisting of 28 amino acids), which are expressed in neurons localized in the hypothalamus of the brain, have been discovered as endogenous ligands for G protein-coupled receptors present mainly in the brain, that is, orexin receptors. (WO1996/34877, Japanese Unexamined Patent Publication Nos. H10-327888, H10-327889, H11-178588, and H10-229887, WO2016/063995, and Sakurai T. et al., Cell, 1998, 92, 573-585.) Orexin receptors include two subtypes: an OX1 receptor (OX1) as a subtype 1 and an OX2 receptor (OX2) as a subtype 2. OX1 selectively binds to OX-A rather than OX-B, and OX2 binds to OX-A as well as to OX-B. Orexin has been determined to stimulate food consumption of rats, suggesting a physiological function of these peptides as a mediator in the central feedback mechanism to regulate feeding behaviors (Sakurai T. et al., Cell, 1998, 92, 573-585).

Orexin has also been observed to regulate the sleep-wake state, and thus may treat narcolepsy as well as insomnia and other sleep disorders (Chemelli R. M. et al., Cell, 1999, 98, 437-451). Furthermore, it has been suggested that orexin signals in the ventral tegmental area in neuroplasticity associated with drug addiction and nicotine addiction play an important role in vivo (S. L. Borgland et al., Neuron, 2006, 49, 589-601 and C. J. Winrow et al., Neuropharmacology, 2010, 58, 185-194). It also has been reported that ethanol addiction is reduced by selectively inhibiting OX2 in an experiment using rats (J. R. Shoblock et al., Psychopharmacology, 2010, 215: 191-203). Furthermore, it also has been reported that in rats, a corticotropin-releasing factor (CRF) related with depression and anxiety disorder is associated with orexin-inductive behaviors, and orexin may play an important role in stress reactions (T. Ida et al., Biochemical and Biophysical Research Communications, 2000, 270, 318-323).

On the other hand, lemborexant (name of the compound: (1R, 25)-2-(((2,4-dimethylpyrimidin-5-yl)oxy)methyl)-2-(3-fluorophenyl)-N-(5-fluoropyridin-2-yl)cyclopropanecarboxamide) is known as a compound having an Orexin receptor antagonistic action and may treat sleep disorders such as insomnia (T. Ida et al., Biochemical and Biophysical Research Communications, 2000, 270, 318-323).

Lemborexant, also known as E2006, has been studied in clinical trials and found to possess advantageous properties, for example, reducing wake after sleep onset, sleep onset latency, and/or improving sleep efficiency. (See, e.g., U.S. Pat. No. 8,268,848 B2 and PCT International Application No. PCT/US2019/039333, the entireties of both of which are incorporated herein by reference.)

Because CYP3A-mediated metabolism is the main clearance pathway for lemborexant, subjects with hepatic impairment may have issues with metabolizing lemborexant, which would affect the PK of lemborexant and lead to contraindications. There is, however, a need for a method for treating patients with moderate hepatic impairment classified in Child-Pugh class B under Child-Pugh Classification. An object of the present disclosure is to provide method for treating insomnia which is effective and safe even if lemborexant is administered to patients with moderate hepatic impairment classified in Child-Pugh class B under Child-Pugh Classification.

In some embodiments, disclosed herein is a method for treating insomnia, comprising: administering orally a dosage form comprising lemborexant or a pharmaceutically acceptable salt thereof to a patient in need thereof at a single daily dose ranging from 5 mg to 10 mg of lemborexant or an equivalent dose of a pharmaceutically acceptable salt thereof, provided that the maximum dose is 5 mg once per day of lemborexant or an equivalent dose of a pharmaceutically acceptable salt thereof when the patient has moderate hepatic impairment classified in Child-Pugh class B under Child-Pugh Classification. In some embodiments, wherein the patient does not have severe hepatic impairment classified in Child-Pugh class C under Child-Pugh Classification.

In some embodiments, disclosed herein is a method for treating insomnia, comprising: administering orally a dosage form comprising lemborexant or a pharmaceutically acceptable salt thereof to a patient in need thereof, wherein a 5 mg dose of lemborexant or an equivalent dose of a pharmaceutically acceptable salt thereof is administered orally to a patient no more than once per night, immediately before going to bed, with at least 7 hours remaining before the planned time of awakening, wherein the dose may be increased to 10 mg of lemborexant or an equivalent dose of a pharmaceutically acceptable salt thereof based on clinical response and tolerability, provided that the maximum dose is 5 mg once per day of lemborexant or an equivalent dose of a pharmaceutically acceptable salt thereof when the patient has moderate hepatic impairment classified in Child-Pugh class B under Child-Pugh Classification. In some embodiments, wherein the patient does not have severe hepatic impairment classified in Child-Pugh class C under Child-Pugh Classification.

In some embodiments, disclosed herein is a method for treating insomnia, comprising: administering orally a dosage form comprising lemborexant or a pharmaceutically acceptable salt thereof to a patient having moderate hepatic impairment classified in Child-Pugh class B under Child-Pugh Classification, wherein the maximum dose is 5 mg once per day of lemborexant or an equivalent dose of a pharmaceutically acceptable salt thereof.

In some embodiments, disclosed herein is a method for treating insomnia in a patient having moderate hepatic impairment classified in Child-Pugh class B under Child-Pugh Classification comprising administering orally once per day a dosage form comprising 5 mg of lemborexant or an equivalent amount of a pharmaceutically acceptable salt thereof.

In some embodiments, disclosed herein is a method for treating insomnia, the method comprising the steps of: determining a hepatic impairment level of a patient under Child-Pugh Classification in a patient; and administering orally a dosage form comprising lemborexant or a pharmaceutically acceptable salt thereof to the patient; wherein a 5 mg dose of lemborexant or an equivalent dose of a pharmaceutically acceptable salt thereof is administered orally to the patient no more than once per night, immediately before going to bed, with at least 7 hours remaining before the planned time of awakening, wherein, when the patient has no hepatic impairment or has mild hepatic impairment classified in Child-Pugh class A under Child-Pugh Classification, the dose may be increased to 10 mg of lemborexant or an equivalent dose of a pharmaceutically acceptable salt thereof based on clinical response and tolerability, and wherein, when the patient has moderate hepatic impairment classified in Child-Pugh class B under Child-Pugh Classification, the maximum dose is 5 mg once per day of lemborexant or an equivalent dose of a pharmaceutically acceptable salt thereof.

According to the present disclosure, insomnia treatment can be effective and safe for patients having moderate hepatic impairment classified in Child-Pugh class B under Child-Pugh Classification.

### BRIEF DESCRIPTION OF THE DRAWINGS

FIG. 1 shows mean (±SD) plasma lemborexant concentration-time profiles after administration of 10 mg lemborexant to healthy control subjects with normal hepatic function, patients with mild hepatic impairment, and patients with moderate hepatic impairment over 12 hours.

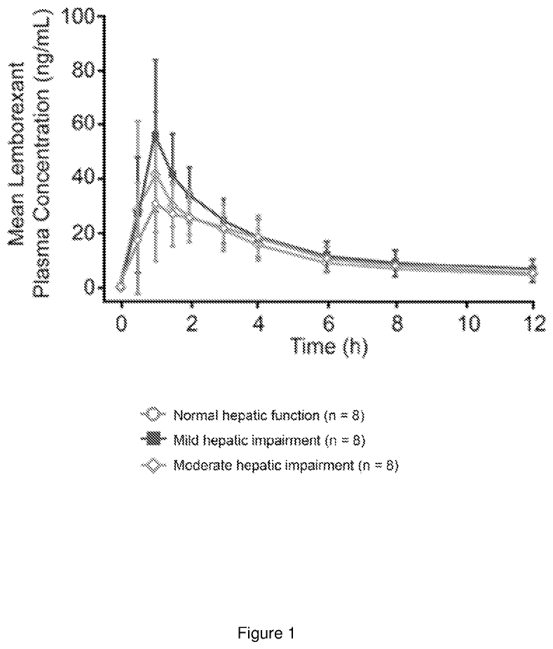

FIG. 2 shows mean (±SD) plasma lemborexant concentration-time profiles after administration of 10 mg lemborexant to healthy control subjects with normal hepatic function, patients with mild hepatic impairment, and patients with moderate hepatic impairment over 312 hours.

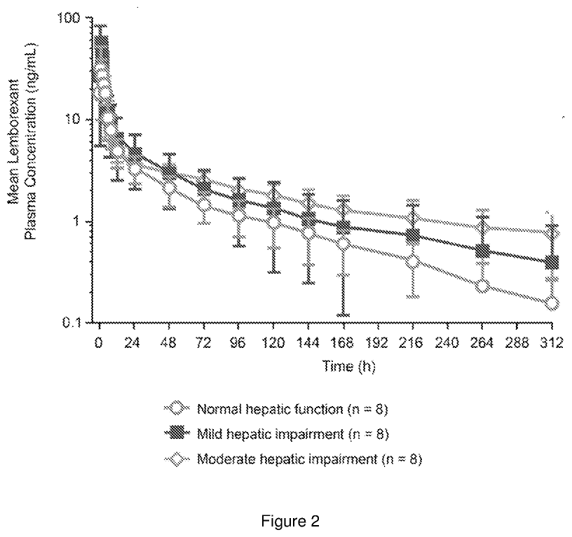

FIG. 3 shows the geometric mean ratios (90% confidence intervals) for patients with mild or moderate hepatic impairment versus healthy control subjects with normal hepatic function.

As used herein, the following definitions shall apply unless otherwise indicated.

As used herein, the term “a” refers to one or more.

As used herein, the term “lemborexant” refers to a compound having the structure:

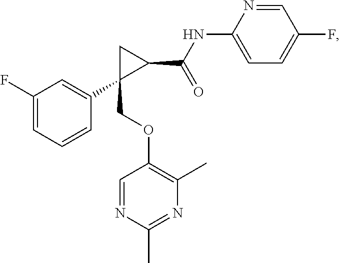 [SMILES: Cc1ncc(OC[C@@]2(c3cccc(F)c3)C[C@H]2C(=O)Nc2ccc(F)cn2)c(C)n1]

also known as (1R,2S)-2-(((2,4-dimethylpyrimidin-5-yl)oxy)methyl)-2-(3-fluorophenyl)-N-(5-fluoropyridin-2-yl)cyclopropanecarboxamide or (1R,2S)-2-(((2,4-dimethylpyrimidin-5-yl)oxy)methyl)-2-(3-fluorophenyl)-N-(5-fluoropyridin-2-yl)cyclopropane-1-carboxamide.
Lemborexant or a pharmaceutically acceptable salt thereof can be prepared by the methods described in WO2012/039371 and WO2013/123240, for example.

As used herein, the term “pharmaceutically acceptable salt” is a salt that retains the desired biological activity of the parent compound and does not impart undesired toxicological effects. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes, et al., J. Pharm. Sci., 2005, 94, 10; and Berge, et al., J. Pharm. Sci., 1977, 66, 1, which are incorporated herein by reference.

In some embodiments, lemborexant or the pharmaceutically acceptable salt thereof is administered in a dosage form. In some embodiments, lemborexant or the pharmaceutically acceptable salt thereof is in a solid dosage form, such as, for example, capsules, granules, lozenges, pellets, pills, powders, suspensions, and tablets.

In some embodiments, the dosage form further comprises at least one additional pharmaceutically acceptable component. In some embodiments, the at least one additional pharmaceutically acceptable component is chosen from pharmaceutically acceptable carriers, pharmaceutically acceptable vehicles, and pharmaceutically acceptable excipients.

The dosage form according to the present disclosure is orally administered to a patient who has insomnia and comprises a dose of lemborexant ranging from 5 mg to 10 mg or an equivalent dose of a pharmaceutically acceptable salt of lemborexant. In some embodiments, the dosage form comprises 5 mg of lemborexant. In some embodiments, the dosage form comprises a pharmaceutically acceptable salt of lemborexant in a dose equivalent to 5 mg of lemborexant. In some embodiments, the dosage form comprises 10 mg of lemborexant. In some embodiments, the dosage form comprises a pharmaceutically acceptable salt of lemborexant in a dose equivalent to 10 mg of lemborexant.

As used herein, the term “pharmaceutically acceptable” means that a carrier, diluent, excipient, or vehicle is compatible with other components of a composition and is nontoxic non-toxic to a subject.

As used herein, the term “pharmaceutically acceptable excipient” means an inactive ingredient used as a vehicle (e.g., water, capsule shell, etc.), a diluent, or a component to constitute a dosage form or pharmaceutical composition comprising a drug such as a therapeutic agent. The term also encompasses an inactive ingredient that imparts cohesive function (e.g., binder), disintegrating function (e.g., disintegrator), lubricant function (e.g., lubricating agent), and/or the other function (e.g., solvent, surfactant, etc.) to the composition.

As used herein, the term “patient” means an animal subject, such as a mammalian subject, and for example, a human being. As used herein, the subject may be of any age. In some embodiments, the subject may be 18 years or older.

As used herein, the terms “treatment” and “treating” refer to an approach for obtaining beneficial or desired results including, but not limited to, therapeutic benefit and/or prophylactic benefit.

As used herein, the term “insomnia” means a disorder defined by the Diagnostic and Statistical Manual of Mental Disorders, 5^{th} Edition (2013; “DSM-V”) having the following diagnostic criteria:
A. A predominant complaint of the subject is dissatisfaction with sleep quantity or quality, associated with one (or more) of the following symptoms:
   1. Difficulty initiating sleep (in children, this may manifest as difficulty initiating sleep without caregiver intervention).
   2. Difficulty maintaining sleep, characterized by frequent awakenings or problems returning to sleep after awakenings (in children, this may manifest as difficulty returning to sleep without caregiver intervention).
   3. Early-morning awakening with inability to return to sleep.
B. The sleep disturbances cause clinically significant distress or impairment in social, occupational, educational, academic, behavioral, or other important areas of functioning.
C. The sleep difficulty occurs at least 3 nights per week.
D. The sleep difficulty is present for at least 3 months.
E. The sleep difficulty occurs despite adequate opportunity for sleep.
F. The insomnia is not better explained by and does not occur exclusively during the course of another sleep-wake disorder (e.g., narcolepsy, breathing-related sleep disorder, circadian rhythm sleep-wake disorder, a parasomnia).
G. The insomnia is not attributable to the physiological effects of a substance (e.g., a drug of abuse, a medication).
H. Coexisting mental disorders and medical conditions do not adequately explain the predominant complaint of insomnia.

The term “insomnia” also means a sleep disorder characterized by symptoms including, but not limited to, difficulty in falling asleep, difficulty in staying asleep, intermittent wakefulness, and/or waking up too early. The term also encompasses daytime symptoms such as sleepiness, anxiety, impaired concentration, impaired memory, and irritability. Types of insomnia suitable for treatment with lemborexant or a pharmaceutically acceptable salt thereof include short-term insomnia and chronic insomnia.

As used herein, “treating insomnia” refers to obtaining beneficial or desired results including, but not limited to, therapeutic benefit and/or prophylactic benefit.

As used herein, the term “Cₘₐₓ” indicates the maximum concentration in the plasma.

As used herein, the term “AUC_{(0-inf)}” indicates the area under the plasma concentration-time curve immediately after the administration of an agent (time 0) to infinity.

“Child-Pugh classification” score is used herein as a marker of degree of hepatic impairment and is assessed according to the criteria in Table 1.

TABLE 1
Criteria for Child-Pugh Classification

| Clinical or | | | |
|---|---|---|---|
| Biochemical | Points Scored for Observed Findings^{a} | | |
| Assessment | 1 | 2 | 3 |
| Albumin (g/dL) | >3.5 | 2.8-3.5 | <2.8 |
| Bilirubin (g/dL) | <2 | 2-3 | >3 |
| PT (seconds | <4 | 4-6 | >6 |
| prolonged) | | | |
| or INR | <1.7 | 1.7-2.3 | >2.3 |
| Ascites | None | Mild/Moderate | Tense |
| | | (diuretic-responsive) | (diuretic-refractory) |
| Encephalopathy | None | 1 or 2 (or precipitant- | 3 or 4 (chronic) |
| | | induced) | |

cps = cycle per second,
INR = international normalized ratio,
PT = prothrombin time.
^{a}Total points of 5-6 was scored as Child-Pugh class A, and total points of 7-9 was scored as Child-Pugh class.
^{b}Encephalopathy grades were scored according to common terminology criteria for adverse events (CTCAE): Grade 0: normal consciousness, personality, neurological examination, and/or electroencephalogram; Grade 1: restless, sleep disturbed, irritable/agitated, tremor, impaired bandwriting, and 5 cps waves; Grade 2: lethargic, time-disoriented, inappropriate, asterixis, ataxia, slow triphasic waves; Grade 3: somnolent, stuporous, place-disoriented, hyperactive reflexes, rigidity, slower waves; Grade 4: unarousable coma, no personality/behavior, decerebrate, slow 2-3 cps delta activity.

In some embodiments, disclosed herein is a method for treating insomnia, comprising: administering orally a dosage form comprising lemborexant or a pharmaceutically acceptable salt thereof to a patient in need thereof at a single daily dose ranging from 5 mg to 10 mg of lemborexant or an equivalent dose of a pharmaceutically acceptable salt thereof, provided that the maximum dose is 5 mg once per day of lemborexant or an equivalent dose of a pharmaceutically acceptable salt thereof when the patient has moderate hepatic impairment classified in Child-Pugh class B under Child-Pugh Classification. In some embodiments, wherein the patient does not have severe hepatic impairment classified in Child-Pugh class C under Child-Pugh Classification.

In some embodiments, disclosed herein is a method for treating insomnia, comprising: administering orally a dosage form comprising lemborexant or a pharmaceutically acceptable salt thereof to a patient in need thereof, wherein a 5 mg dose of lemborexant or an equivalent dose of a pharmaceutically acceptable salt thereof is administered orally to a patient no more than once per night, immediately before going to bed, with at least 7 hours remaining before the planned time of awakening, wherein the dose may be increased to 10 mg of lemborexant or an equivalent dose of a pharmaceutically acceptable salt thereof based on clinical response and tolerability, provided that the maximum dose is 5 mg once per day of lemborexant or an equivalent dose of a pharmaceutically acceptable salt thereof when the patient has moderate hepatic impairment classified in Child-Pugh class B under Child-Pugh Classification. In some embodiments, wherein the patient does not have severe hepatic impairment classified in Child-Pugh class C under Child-Pugh Classification.

In some embodiments, disclosed herein is a method for treating insomnia, comprising: administering orally a dosage form comprising lemborexant or a pharmaceutically acceptable salt thereof to a patient having moderate hepatic impairment classified in Child-Pugh class B under Child-Pugh Classification, wherein the maximum dose is 5 mg once per day of lemborexant or an equivalent dose of a pharmaceutically acceptable salt thereof.

In some embodiments, disclosed herein is a method for treating insomnia in a patient having moderate hepatic impairment classified in Child-Pugh class B under Child-Pugh Classification comprising administering orally once per day a dosage form comprising 5 mg of lemborexant or an equivalent amount of a pharmaceutically acceptable salt thereof.

In some embodiments, disclosed herein is a method for treating insomnia, the method comprising the steps of: determining a hepatic impairment level of a patient under Child-Pugh Classification in a patient; and administering orally a dosage form comprising lemborexant or a pharmaceutically acceptable salt thereof to the patient; wherein a 5 mg dose of lemborexant or an equivalent dose of a pharmaceutically acceptable salt thereof is administered orally to the patient no more than once per night, immediately before going to bed, with at least 7 hours remaining before the planned time of awakening, wherein, when the patient has no hepatic impairment or has mild hepatic impairment classified in Child-Pugh class A under Child-Pugh Classification, the dose may be increased to 10 mg of lemborexant or an equivalent dose of a pharmaceutically acceptable salt thereof based on clinical response and tolerability, and wherein, when the patient has moderate hepatic impairment classified in Child-Pugh class B under Child-Pugh Classification, the maximum dose is 5 mg once per day of lemborexant or an equivalent dose of a pharmaceutically acceptable salt thereof.

No Non-limiting embodiments of the present disclosure include:
Embodiment 1: A method for treating insomnia, comprising:
   administering orally a dosage form comprising lemborexant or a pharmaceutically acceptable salt thereof to a patient in need thereof at a single daily dose ranging from 5 mg to 10 mg of lemborexant or an equivalent dose of a pharmaceutically acceptable salt thereof,
   provided that the maximum dose is 5 mg once per day of lemborexant or an equivalent dose of a pharmaceutically acceptable salt thereof when the patient has moderate hepatic impairment classified in Child-Pugh class B under Child-Pugh Classification.
Embodiment 2: A method for treating insomnia, comprising:
   administering orally a dosage form comprising lemborexant or a pharmaceutically acceptable salt thereof to a patient in need thereof,
   wherein a 5 mg dose of lemborexant or an equivalent dose of a pharmaceutically acceptable salt thereof is administered orally to a patient no more than once per night, immediately before going to bed, with at least 7 hours remaining before the planned time of awakening,
   wherein the dose may be increased to 10 mg of lemborexant or an equivalent dose of a pharmaceutically acceptable salt thereof based on clinical response and tolerability, provided that the maximum dose is 5 mg once per day of lemborexant or an equivalent dose of a pharmaceutically acceptable salt thereof when the patient has moderate hepatic impairment classified in Child-Pugh class B under Child-Pugh Classification.
Embodiment 3: The method of embodiment 1 or 2, wherein the patient does not have severe hepatic impairment classified in Child-Pugh class C under Child-Pugh Classification.
Embodiment 4: A method for treating insomnia, comprising:
   administering orally a dosage form comprising lemborexant or a pharmaceutically acceptable salt thereof to a patient having moderate hepatic impairment classified in Child-Pugh class B under Child-Pugh Classification,
   wherein the maximum dose is 5 mg once per day of lemborexant or an equivalent dose of a pharmaceutically acceptable salt thereof.
Embodiment 5:. A method for treating insomnia in a patient having moderate hepatic impairment classified in Child-Pugh class B under Child-Pugh Classification comprising
   administering orally once per day a dosage form comprising 5 mg of lemborexant or an equivalent amount of a pharmaceutically acceptable salt thereof.
Embodiment 6: A method for treating insomnia, the method comprising the steps of:
   determining a hepatic impairment level of a patient under Child-Pugh Classification in a patient; and
   administering orally a dosage form comprising lemborexant or a pharmaceutically acceptable salt thereof to the patient;
   wherein a 5 mg dose of lemborexant or an equivalent dose of a pharmaceutically acceptable salt thereof is administered orally to the patient no more than once per night, immediately before going to bed, with at least 7 hours remaining before the planned time of awakening,
   wherein, when the patient has no hepatic impairment or has mild hepatic impairment classified in Child-Pugh class A under Child-Pugh Classification, the dose may be increased to 10 mg of lemborexant or an equivalent dose of a pharmaceutically acceptable salt thereof based on clinical response and tolerability, and
   wherein, when the patient has moderate hepatic impairment classified in Child-Pugh class B under Child-Pugh Classification, the maximum dose is 5 mg once per day of lemborexant or an equivalent dose of a pharmaceutically acceptable salt thereof.

### EXAMPLES

The following example illustrates various aspects of the present disclosure and is not to be interpreted as limiting the scope of the disclosure.

A multicenter, single-dose, open-label, parallel-group study in subjects with mild or moderate hepatic impairment and matched (with regard to age [±10 years], sex, and body mass index [BMI, ±20%]) healthy subjects as controls was performed.

The primary objective of the study was to assess the effect of mild and moderate hepatic impairment on the PK of lemborexant after a single-dose administration.

Secondary objectives of the study were: to evaluate the effects of hepatic impairment on the PK of lemborexant metabolites M4, M9, and M10; to evaluate the relationship between the PK parameters of lemborexant and its metabolites and the Child-Pugh classification score, serum albumin, total bilirubin, and PT; and to assess safety and tolerability of lemborexant following a single-dose administration in subjects with mild or moderate hepatic impairment and healthy control subjects.

### Study Phases

The study consisted of 2 phases: Pretreatment and Treatment.

The Pretreatment Phase included 2 study periods: Screening and Baseline (Day −1). The subjects were admitted to the clinical facility on Day −1, remained confined to the clinic until Day 8, and then returned to the clinical facility for additional PK sampling as outpatients until Day 14. In the event of early discontinuation of the subjects, the subjects with Child Pugh class A and B (Cohorts A and B) and the matched healthy controls (Cohort C) were permitted to be replaced.

The Treatment Phase consisted of 1 study period of 14 days.

On Day 1, the subjects were administered a single oral 10 mg dose of lemborexant with approximately 240 mL of water in the morning after an overnight fast. No food was allowed for at least 4 hours postdose. Water was allowed as desired except for 1 hour before and after drug administration.

The blood samples for PK assessments were collected at prespecified intervals up to 312 hours postdose administration. In addition, the blood samples for plasma protein binding assessments of lemborexant were collected from each subject at 2 time points; approximately 1 hour and 24 hours postdose. The subjects were discharged on Day 14 of the study. The end of the study was the date of the last study visit for the last subject.

### Subjects

Subjects were eligible for participation in the study if they met all of the inclusion criteria and none of the exclusion criteria, including:
1. Male or female subjects, aged 18 to 79, at the time of informed consent.
2. BMI between 18 and 40 kg/m² at Screening.
3. Nonsmokers or smokers who smoked 20 cigarettes or less per day.
4. For Cohorts A and B: stable (without any change in disease status for at least 60 days before study screening) hepatic impairment conforming to Child-Pugh classification A or B, respectively (see Table 1), and documented by medical history and a physical examination.
5. For Cohort C: healthy control subjects matched to subjects with hepatic impairment with regard to age (±10 years), sex, and BMI (±20%), and as determined by no clinically significant deviation from normal in medical history, physical examination, ECG, and clinical laboratory determinations.

All subjects were prohibited from having foods, beverages, or supplements (eg, St. John's wort) that affect CYP3A enzyme or transporters (eg, grapefruit-containing foods, vegetables from the mustard green family).

A total of 28 subjects were enrolled; 24 subjects passed screening, were dosed, and completed. All 24 subjects enrolled and dosed (8 subjects per cohort) were included in the Safety and PK Analysis Sets.

Cohort A—Child Pugh Class A: There were 2 female and 6 male subjects. The mean age was 57.0 years.

Cohort B—Child Pugh Class B: There were 2 female and 6 male subjects. The mean age was 61.4 years.

Cohort C—Healthy Control Subjects: There were 3 female and 5 male subjects. The mean age was 56.8 years.

### Assessments

Pharmacokinetics

Blood samples (4 mL each) for PK assessments of lemborexant and its metabolites (M4, M9, and M10) were collected at predose (0 hour), 0.5, 1, 1.5, 2, 3, 4, 6, 8, 12, 24, 48, 72, 96, 120, 144, 168, 216, 264, and 312 hours postdose. In addition, blood samples (12 mL per time point) for protein binding of lemborexant and its metabolites (M4, M9, and M10) were collected at 1 and 24 hours postdose matching the PK sample collection at those time points.

Total mean plasma concentrations of lemborexant and lemborexant metabolites (M4, M9, and M10) were measured by validated liquid chromatography with tandem mass spectrometry (LC-MS/MS). Unbound lemborexant concentrations of lemborexant, M4, M9, and M10 were also measured using a similar validated LC-MS/MS method following equilibrium dialysis.

Safety Assessments

Safety was assessed by monitoring and documenting treat-emergent adverse events (TEAEs), ECGs, vital signs, weight, physical examinations, and clinical laboratory tests (urinalysis, hematology, and blood chemistry).

### Results

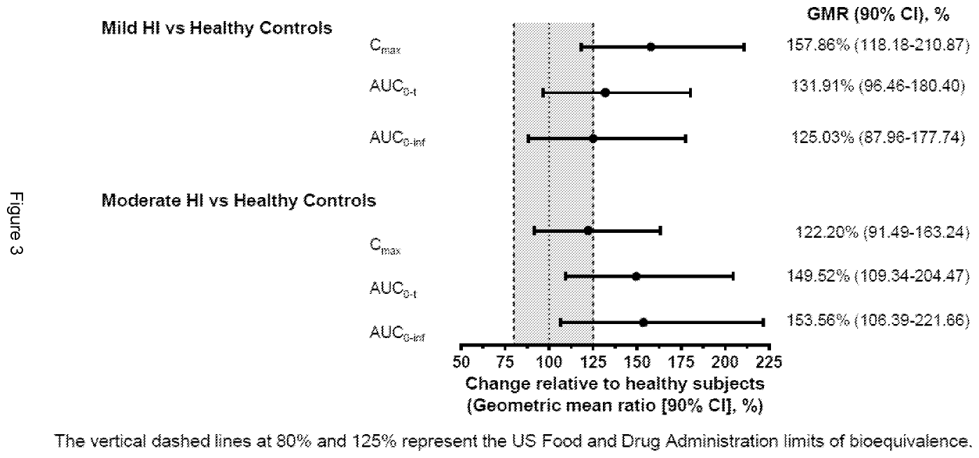

Mild hepatic impairment increased lemborexant Cₘₐₓ and AUC_{(0-inf)} values by 58% and 25% respectively compared to healthy liver function; the effect of moderate hepatic impairment on lemborexant PK was also similar. Lemborexant Cₘₐₓ and AUC_{(0-inf)} values increased 22% and 54%, respectively, in subjects with moderate hepatic impairment compared to healthy subjects. (FIG. 3) Total body clearance of lemborexant decreased by 20% and 35% in subjects with mild and moderate hepatic impairment, respectively, as compared with healthy subjects. Thus, administration of 10-mg lemborexant to subjects with hepatic impairment resulted in an increase in exposure of lemborexant as compared with the administration of 10-mg lemborexant to healthy subjects.

TABLE 2
Geometric Mean (% CV) of Pharmacokinetic Parameters of
Lemborexant after Administration of Lemborexant 10 mg to
Subjects with Normal Hepatic Function, Mild Hepatic
Impairment, or Moderate Hepatic Impairment

| | Normal | Mild | Moderate |
|---|---|---|---|
| | (n = 8) | (n = 8) | (n = 8) |
| Parameter | Geometric Mean (% CV) | | |
| tₘₐₓ (h)^{a} | 1.25 (0.50-4.00) | 1.00(0.50-1.50) | 1.00 (0.50-3.00) |
| Cₘₐₓ (ng/mL) | 39.8 (31.1) | 62.9 (34.9) | 48.7 (37.7) |
| AUC₍₀₋ₜ₎ (h * ng/mL) | 435 (33.2) | 574 (50.7) | 651 (25.6) |
| AUC_{(0-inf)} (h * ng/mL) | 453 (33.9) | 567 (52.0)^{b} | 696 (34.6)^{c} |
| t_{1/2} (h)^{a} | 67.0 (26.9) | 73.7 (43.6)^{b} | 105 (28.5)^{c} |
| CL/F (L/h) | 22.1 (33.9) | 17.6 (52.0)^{b} | 14.4 (34.6)^{c} |
| Vz/F | 2130 (30.1) | 1880 (72.7)^{b} | 2170 (13.5)^{c} |
| fᵤ | 0.0597 (15.3) | 0.0630 (14.2) | 0.0650 (11.4) |
| AUCᵤ (h * ng/mL) | 27.1 (36.8) | 34.9 (52.6)^{b} | 45.1 (42.6)^{c} |
| CLu/F (L/h) | 370 36.8) | 287 (52.6)^{b} | 222 (42.6)^{c} |

^{a}tmax presented as median (range);
^{b}n = 7, terminal rate constant could not be estimated for 1 subject; and
^{c}n = 6, terminal rate constant could not be estimated for 2 subjects.

Lemborexant exposure (based on geometric mean Cₘₐₓ, AUC₍₀₋ₜ₎, and AUC₍₀₋ₜ₎) after a single-dose of lemborexant 10-mg tablet was higher for subjects with mild or moderate hepatic impairment compared with healthy control subjects. The median lemborexant tₘₐₓ was similar across cohorts, ranging from 1.00 to 1.25 h. Geometric mean CL/F decreased with increased hepatic impairment (17.6 L/h for subjects with mild impairment, 14.4 L/h for subjects with moderate hepatic impairment) and was lower than that observed in healthy control subjects (22.1 L/h). A longer geometric mean half-life was observed in subjects with hepatic impairment (73.7 h for subjects with mild hepatic impairment, 105 h for subjects with moderate hepatic impairment) compared to healthy control subjects (67.0 h). The geometric mean lemborexant f values in subjects with mild and moderate hepatic impairment were 0.0630 and 0.0650, respectively, and were comparable to healthy control subjects (0.0597). The trends for higher AUCᵤ and lower CLu/F with mild or moderate hepatic impairment were consistent with the observations for AUC_{(0-inf)} and CL/F.

TABLE 3
Statistical Analysis of the Natural Log-Transformed Systemic Exposure
Parameters of Lemborexant (Mild or Moderate Hepatic Impairment vs. Normal Hepatic Function)

| | | | | | | Ratio | 90% | 90% |
|---|---|---|---|---|---|---|---|---|
| Treatment | | N | N | GeoMean^{a} | GeoMean^{a} | (%)^{b} | CI ^{c} | CI ^{c} |
| (Test Reference) | Parameter | Test | Ref | Test | Ref | (Test/Ref) | Lower | Upper |
| Mild: Normal | Cₘₐₓ | 8 | 8 | 62.9 | 39.8 | 157.86 | 118.18 | 210.87 |
| | (ng/mL) | | | | | | | |
| | AUC₍ₒ₋ₜ₎ | 8 | 8 | 574 | 435 | 131.91 | 96.46 | 180.40 |
| | (h * ng/mL) | | | | | | | |
| | AUC_{(0-inf)} | 7 | 8 | 567 | 453 | 125.03 | 87.96 | 177.74 |
| | (h * ng/mL) | | | | | | | |
| Moderate: Normal | Cₘₐₓ | 8 | 8 | 48.7 | 39.8 | 122.20 | 91.49 | 163.24 |
| | (ng/mL) | | | | | | | |
| | AUC₍ₒ₋ₜ₎ | 8 | 8 | 651 | 435 | 149.52 | 109.34 | 204.47 |
| | (h * ng/mL) | | | | | | | |
| | AUC_{(0-inf)} | 6 | 8 | 696 | 453 | 153.56 | 106.39 | 221.66 |
| | (h * ng/mL) | | | | | | | |

^{a}Geometric Mean for Test and Ref based on Leadt Sqaures Mean of log transformed parameter values;
^{b}Ratio (%) = Geometric Mean (Test)/Geometric Mean (Ref); and
^{c} 90 % CI.

Lemborexant Cₘₐₓ and AUC_{(0-inf)} were 58% and 25% higher, respectively, in subjects with mild hepatic impairment and 22% and 54% higher, respectively, in subjects with moderate hepatic impairment, compared to healthy control subjects.

Lemborexant was extensively metabolized to metabolites M4, M9, and M10. Hepatic impairment had no effect on the metabolite to parent exposure ratios for M4, M9, and M10. Furthermore, the t_{1/2} of M4, M9, and M10 metabolites was unchanged in subjects with mild hepatic impairment and a small 1.5-5 to-2.1-fold increase in subjects with moderate hepatic impairment subjects compared to those in healthy subjects.

TABLE 4
Geometric Mean (% CV) of Pharmacokinetic Parameters
of M4, M9, and M10 after Administration of Lemborexant
10-mg to Subjects with Normal Hepatic Function, Mild
Hepatic Impairment, or Moderate Hepatic Impairment

| | | |
|---|---|---|
| Normal | Mild | Moderate |
| (n = 8) | (n = 8) | (n = 8) |

| | |
|---|---|
| Parameter | Geometric Mean (% CV) |

M4

| | | | |
|---|---|---|---|
| tₘₐₓ (h)^{a} | 2.00 (1.00-4.00) | 1.75 (1.00-4.00) | 1.75 (1.00-4.00) |
| Cₘₐₓ (ng/mL) | 7.97 (33.0) | 7.73 (36.4) | 5.74 (46.7) |
| AUC₍₀₋ₜ₎ (h * ng/mL) | 184 (31.3) | 208 (45.8) | 191 (20.6) |
| AUC_{(0-inf)} (h * ng/mL) | 191 (31.6) | 220 (50.3) | 223 (21.3) |
| t_{1/2} (h)^{a} | 58.5 (63.0) | 60.6 (51.1) | 94.5 (16.7) |
| MPR AUC_{(0-inf)} | 0.405 (11.5) | 0.343 (2.90) | 0.289 (23.4) |

M9

| | | | |
|---|---|---|---|
| tₘₐₓ (h)^{a} | 1.25 (0.50-4.00) | 1.00(0.50-1.50) | 1.00 (0.50-3.00) |
| Cₘₐₓ (ng/mL) | 5.33 (28.9) | 4.49 (45.1) | 3.50 (46.4) |
| AUC₍₀₋ₜ₎ (h * ng/mL) | 88.7 (21.4) | 69.7 (34.9) | 89.6 (23.0) |
| AUC_{(0-inf)} (h * ng/mL) | 92.5 (23.5) | 72.6 (34.7) | 108 (21.6) |
| t_{1/2} (h)^{a} | 44.2 (62.8) | 52.2 (55.7) | 91.5 (24.2) |
| MPR AUC_{(0-inf)} | 0.198 (22.0) | 0.123 (19.0) | 0.144 (17.4) |

M10

| | | | |
|---|---|---|---|
| tₘₐₓ (h)^{a} | 1.25 (0.50-4.00) | 1.00(0.50-1.50) | 1.00 (0.50-3.00) |
| Cₘₐₓ (ng/mL) | 3.71 (34.8) | 3.52 (44.4) | 2.84 (38.4) |
| AUC₍₀₋ₜ₎ (h * ng/mL) | 305 (41.5) | 334 (42.8) | 321 (19.9) |
| AUC_{(0-inf)} (h * ng/mL) | 320 (41.9) | 3.4 (27.7) | 332 (17.9) |
| t_{1/2} (h)^{a} | 60.4 (41.5) | 63. (8 34.4) | 89.2 (24.1) |
| MPR AUC_{(0-inf)} | 0.680 (18.9) | 0.600 (15.3) | 0.502 (20.4) |

^{a}tmax presented as median (range);
^{b}n = 7, terminal rate constant could not be estimated for 1 subject;
^{c}n = 6, terminal rate constant could not be estimated for 2 subjects; and
^{d}n = 5, terminal rate constant could not be estimated for 3 subjects.

For lemborexant metabolites (M4, M9 and M10), each geometric mean Cₘₐₓ was lower in subjects with mild or moderate hepatic impairment compared to healthy control subjects; no apparent differences were observed in the median tmax of each metabolite across cohorts. In general, exposure based on AUC of M4, M9, and M10 was comparable across cohorts and no consistent trends were observed for a change in metabolite exposure with hepatic impairment. Reflecting higher lemborexant exposure (AUC_{(0-inf)}) in subjects with mild or moderate hepatic impairment, the geometric mean metabolite-to-parent ratios (MPR) of AUC_{(0-inf)} decreased with hepatic impairment relative to healthy control subjects.

The relationship between the PK parameters of lemborexant and its metabolites and the Child-Pugh classification score, serum albumin, total bilirubin, MELD score, and PT were explored through scatter plots and linear regression and showed that the effects of mild and moderate hepatic impairment on lemborexant were small and similar No consistent evidence of a relationship between lemborexant PK and hepatic function was observed.

The safety was comparable across mild, moderate, and healthy subjects. No SAEs were reported. A total of 20 (83.3%) subjects experienced a TEAE during the study: 7 (87.5%), 6 (75.0%), and 7 (87.5%) subjects in Class A, Class B, and Healthy Controls, respectively. All TEAEs during the study were mild in severity, and none led to a subject discontinuation. No TEAEs were related to clinically significant abnormalities in laboratory tests, ECGs, vital signs or physical examinations. There were no TEAEs potentially related to cataplexy.

1. A method for treating insomnia, comprising:
administering orally a dosage form comprising lemborexant or a pharmaceutically acceptable salt thereof to a patient in need thereof at a single daily dose ranging from 5 mg to 10 mg of lemborexant or an equivalent dose of a pharmaceutically acceptable salt thereof,
provided that the maximum dose is 5 mg once per day of lemborexant or an equivalent dose of a pharmaceutically acceptable salt thereof when the patient has moderate hepatic impairment classified in Child-Pugh class B under Child-Pugh Classification.

2. A method for treating insomnia, comprising:
administering orally a dosage form comprising lemborexant or a pharmaceutically acceptable salt thereof to a patient in need thereof,
wherein a 5 mg dose of lemborexant or an equivalent dose of a pharmaceutically acceptable salt thereof is administered orally to a patient no more than once per night, immediately before going to bed, with at least 7 hours remaining before the planned time of awakening,
wherein the dose may be increased to 10 mg of lemborexant or an equivalent dose of a pharmaceutically acceptable salt thereof based on clinical response and tolerability, provided that the maximum dose is 5 mg once per day of lemborexant or an equivalent dose of a pharmaceutically acceptable salt thereof when the patient has moderate hepatic impairment classified in Child-Pugh class B under Child-Pugh Classification.

3. The method of claim 1, wherein the patient does not have severe hepatic impairment classified in Child-Pugh class C under Child-Pugh Classification.

4. A method for treating insomnia, comprising:
administering orally a dosage form comprising lemborexant or a pharmaceutically acceptable salt thereof to a patient having moderate hepatic impairment classified in Child-Pugh class B under Child-Pugh Classification,
wherein the maximum dose is 5 mg once per day of lemborexant or an equivalent dose of a pharmaceutically acceptable salt thereof.

5. A method for treating insomnia in a patient having moderate hepatic impairment classified in Child-Pugh class B under Child-Pugh Classification comprising
administering orally once per day a dosage form comprising 5 mg of lemborexant or an equivalent amount of a pharmaceutically acceptable salt thereof.

6. A method for treating insomnia, the method comprising the steps of:
determining a hepatic impairment level of a patient under Child-Pugh Classification in a patient; and
administering orally a dosage form comprising lemborexant or a pharmaceutically acceptable salt thereof to the patient;
wherein a 5 mg dose of lemborexant or an equivalent dose of a pharmaceutically acceptable salt thereof is administered orally to the patient no more than once per night, immediately before going to bed, with at least 7 hours remaining before the planned time of awakening,
wherein, when the patient has no hepatic impairment or has mild hepatic impairment classified in Child-Pugh class A under Child-Pugh Classification, the dose may be increased to 10 mg of lemborexant or an equivalent dose of a pharmaceutically acceptable salt thereof based on clinical response and tolerability, and
wherein, when the patient has moderate hepatic impairment classified in Child-Pugh class B under Child-Pugh Classification, the maximum dose is 5 mg once per day of lemborexant or an equivalent dose of a pharmaceutically acceptable salt thereof.

7. The method of claim 2, wherein the patient does not have severe hepatic impairment classified in Child-Pugh class C under Child-Pugh Classification.
